(19) United States
(12) Patent Application Publication
(10) Pub. No.: US 2010/0274278 A1
(43) Pub. Date: Oct. 28, 2010

(54) ENDOSCOPIC TISSUE GRASPING APPARATUS AND METHOD

(75) Inventors: Richard P. Fleenor, Englewood, CO (US); Robert L. Bromley, El Paso, TX (US)

Correspondence Address: MARSH, FISCHMANN & BREYFOGLE LLP, 8055 East Tufts Avenue, Suite 450, Denver, CO, 80237, US

(73) Assignee: Pare Surgical, Inc., Englewood, CO (US)

(21) Appl. No.: 12/765,709

(22) Filed: Apr. 22, 2010

Related U.S. Application Data
(60) Provisional application No. 61/171,786, filed on Apr. 22, 2009.

(51) Int. Cl. A61B 17/29

(52) U.S. Cl. 606/206; 606/208

(57) ABSTRACT

An improved endoscopic tissue grasping apparatus and method provide for enhanced tissue retention. In one separate aspect, an endoscopic tool may include an improved handle comprising a rachet interface for retaining first and second jaw members at in a closed position at a distal end of the tool. In another separate aspect, a tissue grasper is provided in which opposing first and second jaw members may be effectively locked in a closed position by virtue of an articulating linkage arrangement.

### RELATED APPLICATIONS

This application claims priority to U.S. Provisional Application Ser. No. 61/171,786, filed Apr. 22, 2009, entitled “ENDOSCOPIC TISSUE GRASPING APPARATUS AND METHOD”, the entirety of which is hereby incorporated by reference.

### FIELD OF THE INVENTION

The present invention relates to endoscopic surgical tools, and in particular, to endoscopic tissue grasping apparatus and methods.

### BACKGROUND OF THE INVENTION

A variety of endoscopic tools have been developed to access interior surfaces of bodily organs and other vascular tissue. By way of example, endoscopic ligating and complimentary tools have been developed, as disclosed in U.S. Pat. Nos. 6,554,845, and 6,908,427, hereby incorporated by reference in their entirety.

In conjunction with the use of such endoscopic devices, it is often desirable to maintain the tissue region of interest in a relative stationary position as diagnostic, surgical and/or therapeutic procedures are completed. To date, however, such tissue stabilization has been achieved via the utilization of devices that entail ongoing manipulation of an endoscopic tool by medical personnel throughout the tissue retention time. In turn, such personnel are not able to perform other medical procedures. Further, the approaches utilized to date have entailed the manual application of variable clamping pressures to the tissue region of interest. In this regard, it may be appreciated that the internal tissue region of interest for many endoscopic procedures is quite sensitive and the risk of tissue degradation due to variable gripping pressure is significant, thereby rendering known approaches problematic for many applications.

### SUMMARY OF THE INVENTION

In view of the foregoing, a primary objective is to provide an improved endoscopic tool and method for tissue grasping and tissue retention.

Another objective of the present invention is to provide an improved endoscopic tool and method that may be employed to enhance medical personnel efficiencies attendant to an endoscopic procedure.

Yet another objective of the present invention is to provide an improved endoscopic tool and method that is user-friendly.

An additional objective of the present invention is to provide an improved endoscopic tool that is relatively simple in construction and assembly.

An inventive endoscopic tool is provided for selective tissue grasping and retention. In one feature, the endoscopic tool may include an elongate member, e.g. a flexible elongate member, a tissue grasper located at a distal end of the elongate member, and a handle located at a proximal end of the elongate member.

In one aspect, the handle of the endoscopic tool may include a shaft member and a grip member extending about at least a portion of the shaft member, wherein the shaft member and grip member are disposed for selective relative movement by a user along a handle axis. One of the shaft member and the grip member may include a pawl, while the other of such members may include a rack comprising teeth spaced along the handle axis so as to define a rachet interface.

In turn, the endoscopic tool may be provided so that upon first relative movement between the grip member and shaft member by a user, a first relative movement between opposing portions of first and second jaw members comprising the tissue grasper may be realized, wherein the first and second jaw members may move from an open position to a closed position. Correspondingly, the rachet interface may advantageously retain the opposing portions of the first and second jaw members in the closed position, while maintaining a relatively constant level of tissue grasping force. In the later regard, the degree of closure may be selectively established by a user, then maintained by the rachet interface, wherein the user may selectively affect a desired grasping force that is sufficient to stabilize the tissue free from trauma thereto.

In one approach, the handle axis may be linear, wherein relative movement of the grip member and shaft member of the handle is along such handle axis, and wherein the rachet interface is provided along the same linear handle axis. Such an arrangement yields a compact, user-friendly endoscopic tool which may be provided to facilitate single-hand operation by a user.

In another aspect, one of the pawl and the rack may be provided to be selectively moveable laterally away from the other by a user so as to permit selective second relative movement between the grip member and the shaft member. Such second relative movement may affect second relative movement between the first and second jaw members of the tissue grasper from a closed position to an open position.

In a related aspect, a biasing member may be provided for applying a biasing force to oppose the first relative movement between the grip member and the shaft member. In this regard, the biasing force facilitates the rachet interface engagement of the pawl and rack. In turn, upon relative lateral movement of the pawl and the rack away from one another, the biasing force automatically affects the second relative movement between the grip member and the shaft member, and between the first and second jaw members of the tissue grasper.

An inventive method is also provided for operating an endoscopic tool having an elongate member, a tissue grasper located at a distal end of the elongate member, and a handle located at a proximal end of the elongate member. The method includes the steps of first manually manipulating the handle of the tool to affect first relative movement between a shaft member and a grip member of the handle, wherein one of the grip member and the shaft member includes a pawl and one includes a rack to define a rachet interface therebetween. The method further includes the step of advancing at least one of opposing portions of first and second jaw members comprising a tissue grasper toward the other one, from an open position to a closed position, in mechanical response to the first manually manipulating step, wherein the rachet interface retains the first and second jaw members in the closed position.

In one aspect, the method may include the steps of second manually manipulating the handle to affect second relative movement between the shaft member and the grip member, and retracting at least one of the opposing portions of the first and second jaw members away from the other one, to an open position, in response to the second manually manipulating step. In the later regard, the method may further provide for applying a biasing force to one of the grip member and shaft member, and selectively releasing such biasing force, wherein said retracting step is realized.

In another feature, an inventive endoscopic tool for selective tissue grasping and retention is provided that may include a flexible elongate member and a tissue grasper located at a distal end of the elongate member, wherein flexible elongate member may include a first elongate member and a second elongate member. The first and second elongate members may be disposed for relative movement therebetween.

The tissue grasper may include a first jaw member and a second jaw member. The first jaw member may be disposed for movement between an open position and a closed position in response to a relative movement of distal ends of the first and second elongate members between a first relative position and a second relative position. In turn, tissue is graspable between opposing portions of the first and second jaw members when the first jaw member is in the closed position.

The tissue grasper may also include a first articulating member moveably interconnected to, and moveable relative to a first axis located in fixed spatial relation to, a distal end of a predetermined one of the first and second elongate members. The first articulating member may be moveable along a first path between a first articulated position and a second articulated position relative to the first axis in response to the relative movement of the distal ends of the first and second elongate members. In turn, the first articulating member may be moveably interconnected to the first jaw member so as to affect movement of the first jaw member between the open position and the closed position in response to relative movement of the distal ends of the first and second elongate members, and so that when the distal ends of the first and second elongate members are disposed in the second relative position a separating force applied to the first jaw member will not affect an application of force to the first articulating member in a direction along the first path (e.g., from the second articulated position to the first articulated position). Accordingly, the first jaw member may be effectively locked in a closed position such that a separating force acting on the first jaw member does not result in the first jaw member opening from the closed position.

As may be appreciated, such locking feature may advantageously provide for reduced variability in tissue grasping pressure. Further, such feature may yield enhanced personnel efficiencies by facilitating tissue grasping free from the continuous application of force by a user (e.g., free from grasping a handle).

In one aspect, the distal ends of the first and second elongate members may be disposed for relative axial movement. As such, the first and second relative positions of the distal ends of the first and second elongate members may be axially offset.

In another aspect, the first articulating member may be pivotally interconnected at the first axis to the distal end of the above-noted, predetermined one of first and second elongate members. In turn, at least a portion of the first path of the first articulating member between the first and second articulated positions may be arcuate. Additionally, the first jaw member may be pivotally interconnected to the first articulating member at a second axis offset from the first axis. The second axis may be located at a first offset position, or first angular position, with respect to the first axis, when the first articulating member is in the first articulated position. Correspondingly, the second axis may be located in a second offset position, or second angular position, with respect to the first axis, when the first articulating member is in the second articulated position. As may be appreciated, the first and second offset positions may be offset.

Further, the first jaw member may be moveably interconnected to, and moveable relative to a third axis located in fixed spatial relation relative to, the distal end of the above-noted, predetermined one of the first and second elongate members. The first axis and the third axis may be offset. When the first jaw member is in the closed position, the first axis and the second axis, and the first axis and the third axis, may define an included angle at the first axis of at least 90 degrees. As such, any force acting on the first articulating member in response to a separating force applied to the first jaw member does not result in a force in a direction along the first path between the second and first articulated positions of the first articulating member. In turn, undesired movement of the first jaw member between the closed and open positions in response to a separating force may be avoided.

In some implementations the first axis and the third axis may be disposed orthogonal to, and intersecting, a longitudinal axis of the distal end of the above-noted one of the first and second elongate members. In this regard, the first and second elongate members may be disposed so that, upon relative axial movement thereof, aligned, efficient and predictable responsive movement of the first articulating member and first jaw member may be realized.

Further, the first jaw member may be pivotally interconnected at the third axis to the distal end of the one of the first and second elongate members. In this regard, the tissue grasper may also include a first shaft member disposed on the third axis that may be fixedly interconnected to the distal end of one of the first and second elongate members. Also, a slot formed in a portion of the first jaw member may be provided for receiving the first shaft member therein. The first shaft member may be disposed for relative movement to and within the slot when the first jaw member is moved between the open and closed positions.

In still another aspect, the tissue grasper may include a first linkage member moveably interconnected at a fourth axis to the first articulating member and moveably interconnected to, and moveable relative to a fifth axis located in fixed spatial relation relative to, a distal end of the other one of the distal ends of the first and second elongate members (i.e., not the above-noted predetermined end). The first linkage member may be pivotally interconnected to the first articulating member at the fourth axis. The first linkage member may be pivotally interconnected to the distal end of the other one of the first and second elongate members at the fifth axis.

In some implementations, the first axis, the third axis, and the fifth axis may be arranged orthogonally relative to, and intersecting, a longitudinal axis of the distal end of one of the first and second elongate members. In this regard, the first and second elongate members may be disposed so that, upon relative axial movement thereof, aligned, efficient and predictable response movement of the first articulating member first jaw member and first linkage member may be realized.

In still another aspect of the tissue grasper, the second jaw member may be disposed for movement between an open position and a closed position in response to the relative movement of the distal ends of the first and second elongate members. In this regard, the tissue grasper may also include a second articulating member moveably interconnected at the first axis to the distal end of one of the first and second elongate members. The second articulating member may be moveable along a second path between a third articulated position, or third angular position with respect to the first axis, and fourth articulated position, or angular position, relative to the first axis in response to the relative movement of the distal ends of the first and second elongate members.

Furthermore, the second articulating member may be moveably interconnected to the second jaw member as to affect the movement of the second jaw member between the open position and the closed position thereof in response to the relative movement of the distal ends of the first and second elongate members, and so that when the first and second the distal ends of the first and second elongate members are disposed in the second relative position a separating force applied to the second jaw member will not affect an application of force to the second articulating member in a direction along the second path (e.g., from the fourth articulated position to the third articulated position).

In another aspect, the second articulating member may be pivotally interconnected at the first axis to the distal end of the above-noted, predetermined one of the first and second elongate members. In turn, at least a portion of the second path of the second articulating member between the third and fourth articulated positions may be arcuate.

The second jaw member may be pivotally interconnected to the second articulating member at a sixth axis offset from the first axis. The sixth axis may be located at a third offset position, or third angular position, with respect to the first axis, when the second articulating member is in the third articulated position. Correspondingly, the sixth axis may be located in a fourth offset position, or fourth angular position, with respect to the first axis, when the second articulating member is in the fourth articulated position. As may be appreciated, the third and fourth offset positions may be offset.

The second jaw member may be pivotally interconnected to, and moveable relative to, the third axis. When the second jaw member is in the closed position, the first axis and the sixth axis and the first axis and the third axis may define an included angle at the first axis of at least 90 degrees. As such, any force acting on the second articulating member in response to a separating force applied to the second jaw member does not result in a force in a direction along the second path between the fourth and third articulated positions of the second articulating member. In turn, undesired movement of the second jaw member between the closed and open positions in response to a separating force may be avoided.

In another aspect, the tissue grasper may also include a second slot formed in a portion of the second jaw member for receiving the first shaft member therein. The first shaft member may be disposed for relative movement to and within the second slot when the second jaw member is moved between the open and closed positions.

Also, a second linkage member may be moveably interconnected (e.g., pivotally interconnected) to the second articulating member at a seventh axis and moveably interconnected to the distal end of the other one of the distal ends of the first and second elongate members (i.e., not the above-noted predetermined one).

An additional inventive method is also provided for operating an endoscopic tool comprising a flexible elongate member and a tissue grasper located at a distal end of the flexible elongate member wherein the tissue grasper includes first and second jaw members for selective tissue grasping and retention. The method includes moving at least a predetermined one of a first elongate member and a second elongate member of the flexible elongate member relative to the other one between a first relative position and a second relative position. Also, the method includes positioning a first articulating member, moveably interconnected to, and moveable relative to a first axis located in fixed spatial relation relative to, a distal end of one of the first and second elongate members, along a first path between a first articulated position and a second articulated position relative to the first axis in response to the moving step. The method further includes locating the first jaw member, moveably interconnected to the first articulating member, between an open position and a closed position so as to grasp tissue between the first and second jaw members in response to the moving step, wherein when the distal ends of the first and second elongate member are disposed in the second relative position, a separating force applied to the first jaw member will not affect an application of force to the first articulating member in a direction along the first path.

In one aspect, the moving step may include axially moving a distal end of the above-noted one of the first and second elongate members to a distal end of the other one of the first and second elongate members. In turn, the first relative position and the second relative position may be axially offset.

Additionally, the positioning step may include pivoting the first articulating member relative to the first axis. In turn, at least a portion of the first path of the articulating member between the first and second articulated positions may be arcuate.

Further, the locating step may include pivoting the first jaw member with respect to the first articulating member at a second axis offset from the first axis. The second axis may be located at a first offset position when the first articulating member is in the first articulated position, and wherein the second axis is located in a second offset position when the first articulating member is in the second articulated position. As may be appreciated, the first and second offset positions may be offset.

In another aspect, the locating step may also include situating the first jaw member relative to a third axis located in fixed spatial relation relative to the distal end of the one of the first and second elongate members. The first axis and the third axis may be offset. Accordingly, when the first jaw member is in the closed position, the first axis and the second axis may define an included angle at the first axis of at least 90 degrees.

In yet another aspect, the method may include translating movement of the above-noted, predetermined one of the first and second elongate members in the moving step to the first articulating member to complete the positioning step with a linkage member. The linkage member may be interconnected at a fourth axis to the first articulating member and moveably interconnected to, and moveable relative to a fifth axis located in fixed spatial relation relative to, a distal end of the other one of the distal ends of the first and second elongate members. Additionally, the method may include maintaining the first axis, the third axis, and the fifth axis in orthogonal intersection with a longitudinal axis of the distal end of the one of the first and second elongate members.

In yet another aspect, the method may include disposing, or moving, a second articulating member moveably interconnected to, and moveable relative to the first axis is a fixed spatial relation relative to, a distal end of one of the first and second elongate members, along a second path between a third articulated position and a fourth articulated position relative to the first axis in response to the moving step. In this regard, the method may also include orienting the second jaw member, moveably interconnected to the second articulating member, between an open position and a closed position so as to grasp tissue, in response to the moving step, wherein when the distal ends of the first and second elongate members are disposed in the second relative position, a separating force applied to the second jaw member will not affect an application of force to the second articulating member in a direction along the second path.

In one approach, the orienting step may include pivoting the second jaw member with respect to the second articulating member at a sixth axis offset from the first axis. The sixth axis may be located at a third offset position when the second articulating member is in the third articulated position, and the sixth axis may be located in a fourth offset position when the second articulating member is in the fourth articulated position. As may be appreciated, the third and fourth offset positions are offset.

The orienting step may also include situating the second jaw member relative to the third axis that is located in a fixed spatial relation relative to the distal end of the above-noted predetermined one of the first and second elongate members. When the second jaw member is in the closed position, the first axis and the sixth axis may define an included angle at the first axis of at least 90 degrees.

In yet another aspect, the method may include translating movement of the above-noted, predetermined one of the first and second elongate members in the step to the second articulating member to complete the disposing step with a second linkage member interconnected at a seventh axis to the second articulating member.

In another feature, an inventive endoscopic tool for selective tissue grasping and retention is provided that may include a flexible elongate member having a tissue grasper located at a distal end of the elongate member. The elongate member may include a first elongate member and a second elongate member. The first and second elongate members may be disposed for relative movement therebetween;

The tissue grasper may include a first jaw member and a second jaw member. At least the first jaw member may be disposed for movement between an open position and a closed position in response to a relative movement of distal ends of the first and second elongate members between a first relative position and a second relative position. Tissue may be graspable between opposing portions of the first and second jaw members when the first jaw member is in the closed position.

The tissue grasper may also include a first articulating member moveably interconnected to, and moveable relative to a first axis located in fixed spatial relation relative to, a distal end of one of the first and second elongate members, in response to the relative movement of the distal ends of the first and second elongate members. The first jaw member may be moveably attached to, and moveable relative to a third axis in a fixed spatial relation relative to the distal end of one of the first and second elongate members and offset from the first axis. The first articulating member may be moveably interconnected to the first jaw member at a second axis offset from the first axis so as to affect the movement of the first jaw member between the open position and the closed position in response to the relative movement of the distal ends of the first and second elongate members. In this regard, a first length between the second axis and the third axis is longer when in the closed position than in the open position.

In another aspect, a first distance from a distal end of the first jaw member to the third axis may be shorter when in the closed position than the open position. Accordingly, a ratio between the first length and the first distance may be greater when in the closed position than in the open position.

In yet another aspect, a first shaft member may be disposed on the third axis and fixedly interconnected to the distal end of the one of the first and second elongate members. Also, a first slot formed in a portion of the first jaw member for receiving the first shaft member therein. The first shaft member may be disposed for relative movement to and within the first slot when the first jaw member is moved between the open and closed positions.

In another aspect, the first length between the second axis and the third axis may define a first lever arm and the first distance between the distal end of the first jaw member and the third axis may define a second lever arm. Relative movement between the first elongate member and the second elongate member may result in transmission of a force between the first lever arm and the second lever arm.

In still another aspect, a first linkage member may be moveably interconnected at a fourth axis to the first articulating member and moveably interconnected to, and moveable relative to a fifth axis located in fixed spatial relation relative to, a distal end of the other one of the distal ends of the first and second elongate members.

In another aspect, the second jaw member may be disposed for movement between an open position and a closed position in response to the relative movement of the distal ends of the first and second elongate members.

In this regard, the tissue grasper may include a second articulating member that may be moveably interconnected at the first axis to the distal end of one of the first and second elongate members in response to the relative movement of the distal ends of the first and second elongate members. The second jaw member may be moveably attached to, and moveable relative to the third axis. The second articulating member may be moveably interconnected to the second jaw member at a sixth axis offset from the first axis so as to affect the movement of the first jaw member between the open position and the closed position in response to the relative movement of the distal ends of the first and second elongate members. In turn, a second length between the sixth axis and the third axis may be longer when in the closed position than in the open position.

In one aspect, a second distance from a distal end of the second jaw member to the third axis may be shorter when in the closed position than the open position. Also, a ratio between the second length and the second distance may be greater when in the closed position than in the open position.

In another aspect, a second slot may be formed in a portion of the second jaw member for receiving the first shaft member therein. The first shaft member may be disposed for relative movement to and within the second slot when the first second member is moved between the open and closed positions.

In another aspect, the second length between the sixth axis and the third axis may define a third lever arm and the second distance between the distal end of the second jaw member and the third axis may define a fourth lever arm. Relative movement between the first elongate member and the second elongate member may result in transmission of a force between the third lever arm and the fourth lever arm.

In another aspect, a second linkage member may be moveably interconnected at a seventh axis to the second articulating member and may be moveably interconnected to, and moveable relative to the fifth axis.

An inventive method is also provided for operating an endoscopic tool having an elongate member, a tissue grasper located at a distal end of the elongate member. The tissue grasper may include first and second jaw members for selective tissue grasping and retention therebetween. The method may include moving at least one of a first elongate member and a second elongate member of the flexible elongate member relative to the other one between a first relative position and a second relative position. The method may also include positioning a first articulating member moveably interconnected to, and moveable relative to a first axis located in fixed spatial relation relative to, a distal end of one of the first and second elongate members along a first path between a first articulated position and a second articulated position relative to the first axis in response to the moving step. Further still, the method may include locating the first jaw member, moveably interconnected to the first articulating member at a second axis offset from the first axis and moveably interconnected to, and moveable with respect to, a third a third axis in a fixed spatial relation relative to the distal end of one of the first and second elongate members and offset from the first axis, between an open position and a closed position so as to grasp tissue between the first and second jaw members in response to the moving step. In turn, a first length between the second axis and the third axis is greater in the closed position than in the open position.

In one aspect, a first distance between a distal end of the first jaw member and the third axis may be shorter when in the closed position than in the open position. The method may involve increasing a ratio between the first length and the first distance when moving from the open position to the closed position. The method may involve decreasing a ratio between the first length and the first distance when moving from the closed position to the open position.

In another aspect, the method may include orienting a first slot formed in a portion of the first jaw member with respect to a first shaft member disposed on the third axis in response to the moving step such that the first distance between the distal end of the first jaw member and the third axis is shorter when in the closed position than in the open position.

In still another aspect, the method may include disposing a second articulating member moveably interconnected to, and moveable relative to the first axis is a fixed spatial relation relative to, a distal end of one of the first and second elongate members, along a second path between a third articulated position and a fourth articulated position relative to the first axis in response to the moving step. The method may also include situating the second jaw member, moveably interconnected to the second articulating member at a fourth axis offset from the first axis and moveably interconnected to, and moveable with respect to, the third axis between an open position and a closed position so as to grasp tissue between the first and second jaw members in response to the moving step. A second length between the fourth axis and the third axis may be greater in the closed position than in the open position.

In another aspect, a second distance between a distal end of the second jaw member and the third axis may be shorter when in the closed position than in the open position. The method may include increasing a ratio between the second length and the second distance when moving from the open position to the closed position. The method may include decreasing a ratio between the second length and the second distance when moving from the closed position to the open position.

In another embodiment, the method may include disposing a second slot formed in a portion of the second jaw member with respect to the first shaft member disposed on the third axis in response to the moving step such that the second distance between the distal end of the first jaw member and the third axis is shorter when in the closed position than in the open position.

Any of the embodiments, arrangements, or the like discussed herein may be used (either alone or in combination with other embodiments, arrangements, or the like) with any of the disclosed aspects. Any feature disclosed herein that is intended to be limited to a “singular” context or the like will be clearly set forth herein by terms such as “only,” “single,” “limited to,” or the like. Merely introducing a feature in accordance with commonly accepted antecedent basis practice does not limit the corresponding feature to the singular (e.g., indicating that a member includes “a pivot member” alone does not mean that the container includes only a single pivot member). Moreover, any failure to use phrases such as “at least one” also does not limit the corresponding feature to the singular (e.g., indicating that a member includes “a pivot member” alone does not mean that the container includes only a single pivot member). Use of the phrase “at least generally,” “at least partially,” or the like in relation to a particular feature encompasses the corresponding characteristic and insubstantial variations thereof. Finally, a reference of a feature in conjunction with the phrase “in one embodiment” does not limit the use of the feature to a single embodiment.

### BRIEF DESCRIPTION OF THE DRAWINGS

FIG. 1 is a perspective view of one embodiment of an endoscopic tool illustrating a handle, elongate member, and tissue grasper thereof in disconnected relation for purposes of illustration.

FIG. 2 is an exploded view of the handle of the endoscopic tool embodiment illustrated in FIG. 1.

FIG. 3A is a side view of the endoscopic tool embodiment of FIG. 1 with the handle, elongate member and tissue grasper thereof shown in interconnected relation.

FIG. 3B is a side cross-sectional view of the endoscopic tool embodiment shown in FIG. 3A, such cross-sectional view taken along cut line AA of FIG. 3A.

FIG. 4A is a side view of the endoscopic tool embodiment shown in FIG. 3A with a grip member of the handle shown in an advance positioned relative to a shaft member of the handle.

FIG. 4B is a side view corresponding with the endoscopic tool embodiment shown in FIG. 3A with the grip member of the handle shown in a retracted position relative to the shaft member thereof to affect tissue grasping.

FIG. 4C is side view corresponding with the endoscopic tool environment shown on FIG. 3A with first and second members of the grip member advanced relative to one another to affect automatic repositioning of the grip member from the retracted position of FIG. 4B to the advanced position of FIG. 4A.

FIG. 5A is a side view of a tissue grasper embodiment interconnected to the distal end of the elongate member of the endoscopic tool embodiment shown in FIG. 1.

FIG. 5B is a top view of the tissue grasper embodiment shown in FIG. 5A as interconnected to the distal end of the elongate member of the endoscopic tool embodiment shown in FIG. 1.

FIG. 5C is a perspective view of the tissue grasper embodiment of FIG. 5A.

FIG. 5D is an exploded view of the tissue grasper embodiment shown in FIG. 5C.

FIGS. 6A, 6B, 6C, 6D, 6E, 6F and 6G illustrate the tissue grasper embodiment of FIG. 5A, with modified jaw surfaces, and with opposing jaw members progressing from a closed position to an open position.

FIGS. 6H, 6I, 6J and 6K illustrate the tissue grasper embodiment of FIG. 5A, with modified jaw surfaces, and with jaw opposing members progressing from an open position to a closed position.

FIG. 7A is an alternate view of the tissue grasper embodiment shown in FIGS. 6A and 6K with additional reference features.

FIG. 7B is an alternative view of the tissue grasper embodiment shown in FIG. 6F, with additional reference features.

FIG. 8A is an alternate view of the tissue grasper embodiment shown in FIG. 7A.

FIG. 8B is an alternate view of the tissue grasper embodiment shown in FIG. 7B.

### DETAILED DESCRIPTION

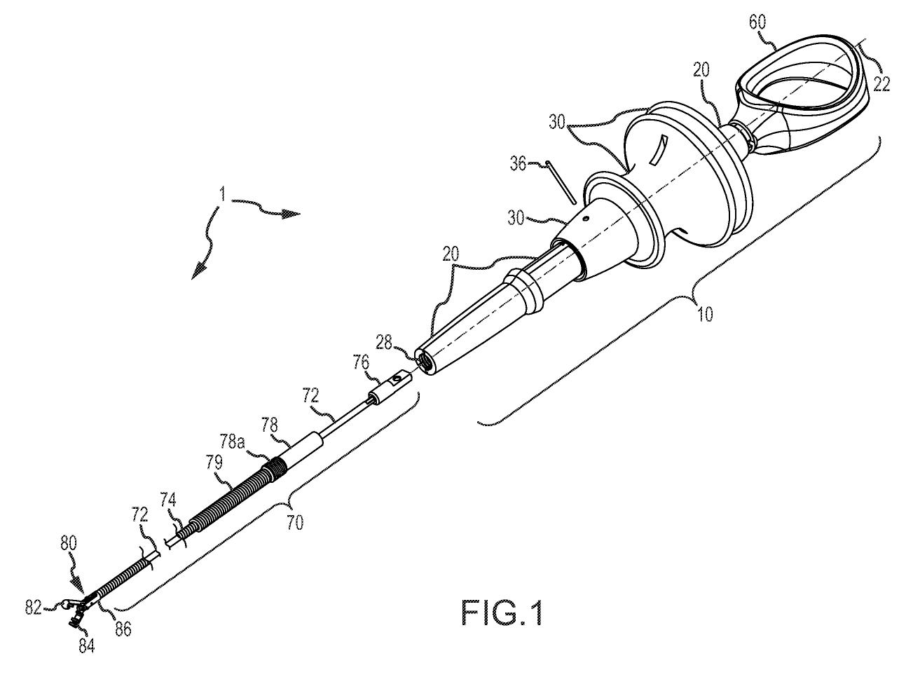

FIG. 1 illustrates one embodiment of an endoscopic tool 1 comprising a handle 10, an elongate member 70 and a tissue grasper 80. The handle 10 is interconnectable to a proximal end of the elongate member 70, and the tissue grasper 80 is interconnectable to a distal end of the elongate member 70.

The handle 10 may include a shaft member 20 and a grip member 30 that may extend at least partially around the shaft member 20. In the illustrated embodiment, the shaft member 20 may be provided to extend through an aperture of the grip member 30 that extends along a handle axis 22. In turn, a proximal end member 60 may be fixedly interconnected to a proximal end of the shaft member 20. As will be further described, the grip member 30 and shaft member 20 may be provided for relative movement therebetween by a user. By way of example only, the grip member 30 and shaft member 30 may be of molded plastic construction (e.g. ABS plastic, nylon plastic, etc.)

The elongate member 70 may be of a flexible construction and may include an inner member 72 (e.g. stainless steel wire) that extends through a tubular outer member 74 (e.g. tightly wound stainless steel spring), wherein the inner member 72 and outer member 74 are disposed for relative movement therebetween. In the illustrated embodiment, a proximal end of the inner member 72 may be provided with a connector 76 having an opening therethrough for interconnection to the grip member 30 via a pin 36 that extends through the grip member 30 and connector 76, wherein co-movement of inner member 72 and grip member 30.

The tissue grasper 80 may comprise opposing first and second jaw members 82 and 84 (e.g. of a metal construction). The first and second jaw members 82 and 84 may be provided with opposing distal portions disposed for selective relative movement between an open position, as shown in FIG. 1, and a closed position in which the opposing portions may be positioned in juxtaposed relation for tissue retention therebetween.

In this regard, the first and second jaw members 82 and 84 may be interconnected to the inner member 72 of the elongate member 70, wherein movement of the inner member 72 affects movement of the first and second jaw members 82 and 84. The tissue grasper 80 may further include a yolk member 86 fixedly interconnected to the outer member 74 of the elongate member 70, as will be further described.

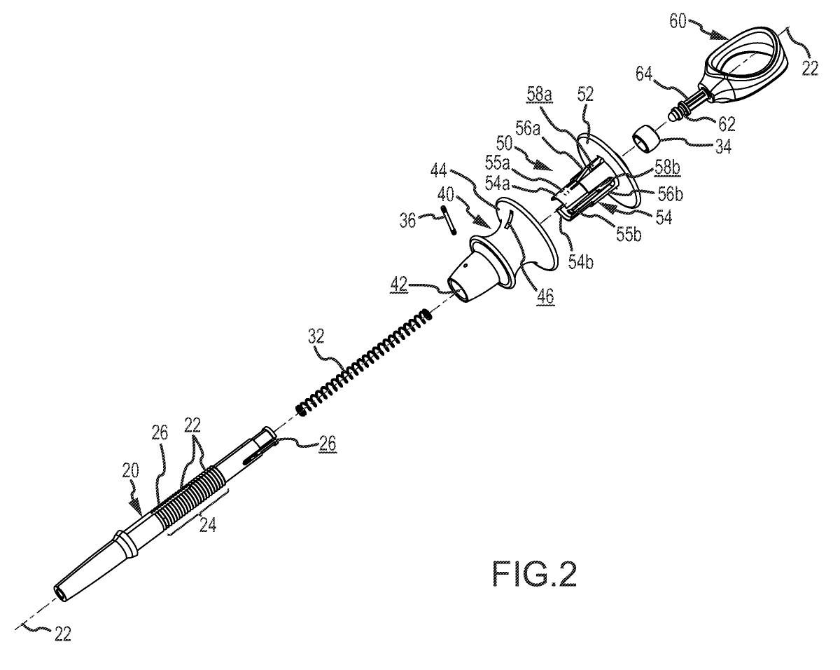

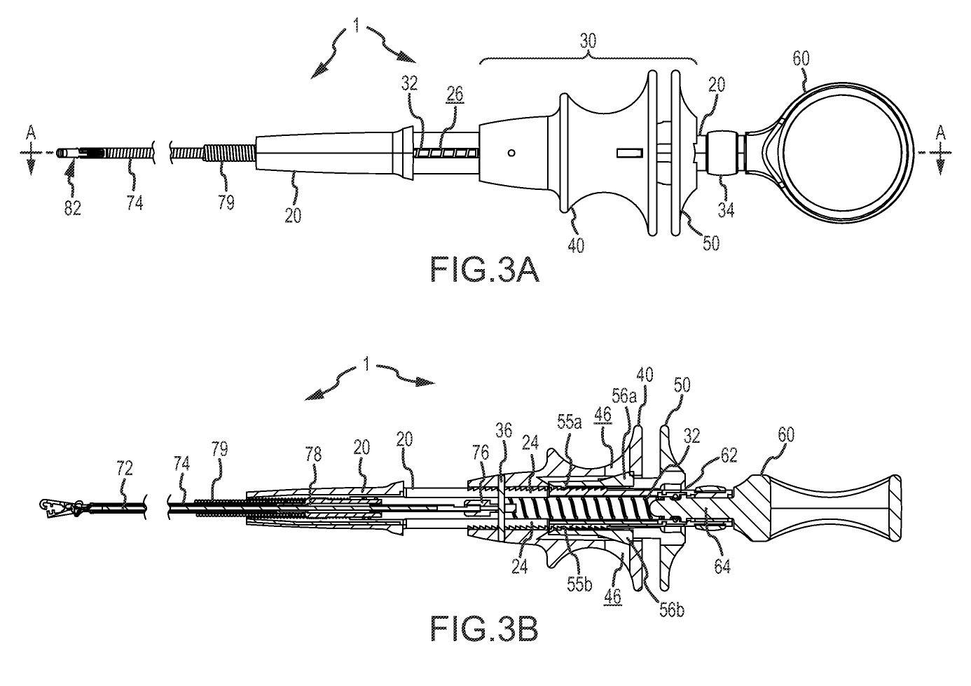

Reference is now made to FIG. 2 and FIGS. 3A and 3B. The shaft member 20 may include a plurality of teeth 22 spaced along a length of an outer surface of the shaft member 20 to define a rack 24. In one approach, rack 24 may be defined by an insert molded metal portion. In the illustrated embodiment, the teeth 22 may each extend about an annular periphery of the shaft member 20. The shaft member 20 may further include an elongate slot 26 for receipt of the above-noted pin 36 therethrough. As will be further described, the pin 36 may move within and along the elongated slot 26 during relative movement of the shaft member 20 and grip member 30.

With further reference to FIG. 2, the grip member 30 may comprise a first member 40 and a second member 50. The first member 40 may include a central aperture 42 that extends therethrough and a laterally extending flange portion 44 near a proximal end thereof. In turn, the second member 50 may include a laterally extending flange portion 52 and a longitudinally extending connector portion 54 that is interconnectable within the aperture 42 of the first member 40. The connector portion 54 may include a plurality of legs extending away from the flange portion 52 for interconnected positioning within the aperture 42. In the illustrated embodiment, two opposing leg members 54a and 54b are illustrated.

Each of the leg members 54a and 54b may be provided with a corresponding connector tab 56a and 56b, respectively, each of which are cantilevered within a corresponding window 58a and 58b, respectively. The connector tabs 56a and 56b may each comprise an outer surface that tapers outwardly away from a distal end to a proximal end thereof. In turn, upon insertion of the connector portion 54 into the aperture 42 of the first member 40, the cantilevered tabs 56a and 56b may elastically deflect inward and then back outward as tabs 56a and 56b advance into snap-fit engagement corresponding apertures 46 of the first member 40. As will be further described, each of the leg members 54a and 54b may each be further provided with a corresponding inwardly-extending, tooth-shaped pawl 55a (shown in phantom lines in FIG. 2) and 55b respectively, located in aligned distal relation with distal ends of the cantilevered tabs 56a and 56b, respectively. With further reference to FIG. 2, the handle 10 may further comprise a biasing member 32 (e.g. a metal spring) for positioning within the shaft member 20 and grip member 30. In the illustrated embodiment upon assembly, a proximal end of the biasing member 32 may be positioned within a corresponding retention notch 62 provided on a stem 64 of the proximal end member 60. A distal end of the biasing member 32 may abut pin 36.

When assembled, the stem 64 of the proximal end member 60 may be inserted into a proximal end portion of the shaft member 20. To facilitate such insertion, the proximal end portion of the shaft member 20 may include one or more slits 26 extending distally from the proximal end to allow for elastic deformation of the proximal end portion of the shaft member during receipt of the stem 64 therewithin. After insert positioning of the stem 64 within the proximal end portion of the shaft member 20, a resilient retention band 34 may be disposed in overlapping relation to the proximal end portion of the shaft member 20 and stem 64 of the proximal end portion member 60 to maintain an axially fixed relation therebetween. In the illustrated arrangement, the proximal end member is axially fixed relative to shaft member 20 that may rotate to an extent relative thereto.

With further reference to FIGS. 1, 3A and 3B, the elongate member 70 may include an interconnect member 78 disposed about a length of the inner member 72 near the proximal end thereof. In turn, the interconnect member 78 may be located within a distal end portion of the shaft member 20 upon assembly, wherein an outer threaded surface portion 78a may rotatively engage and interconnect to an inner threaded surface portion 28 of shaft member 20.

The elongate member 70 may also optionally include a strain relief member 79 (e.g. tightly wound stainless steel spring) that is positioned about a length outer member 74 at the interface of outer member 74 and shaft member 20. In this regard, the strain relief member 79 may address bending forces that may be applied to elongate member 70 during use.

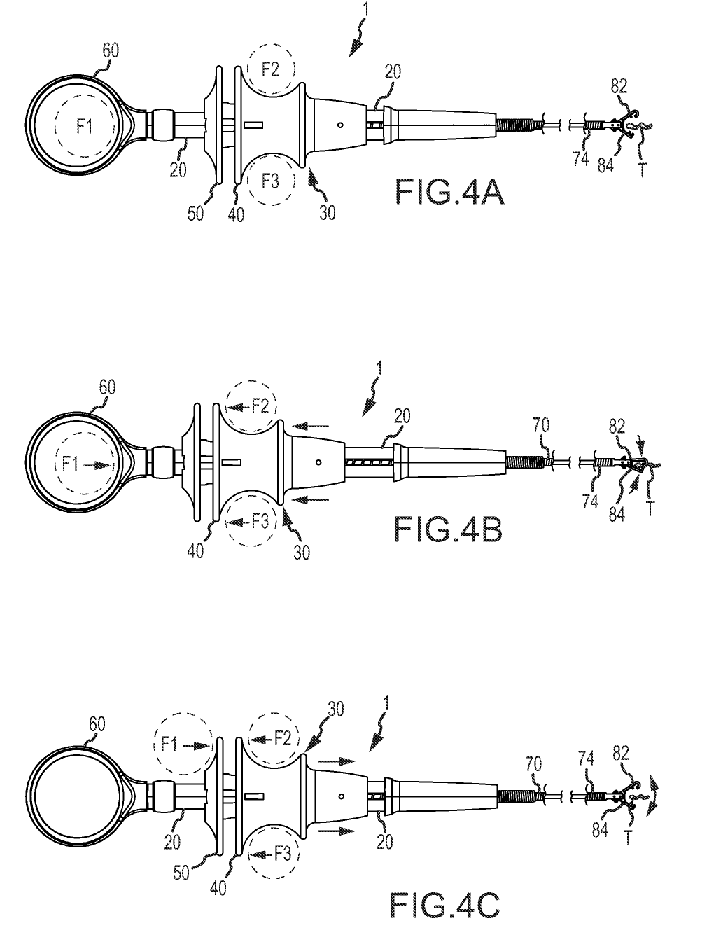

Operation of the endoscopic tool 11 will now be described with reference to FIGS. 4A-4C and FIG. 3B. FIG. 4A shows handle 10 with grip member 30 in a first advanced position relative to the shaft member 20. In the first position the first and second jaw members 82 and 84 are in an open relative position for locating tissue T therebetween. For such tissue locating, the tool 1 may be manipulated via fingers F1, F2 and F3 of a user positioned as shown.

FIG. 4B illustrates retraction of the grip member 30 relative to the shaft 20 so as to close the first and second jaw members 82 and 84 to a closed position with tissue T grasped therebetween. For such retraction, the tool may be manipulated via positioning of fingers F1, F2 an F3 as shown, wherein finger F1 may be advanced relative to fingers F2 and F3 and/or wherein fingers F2 and F3 may be retracted relative to finger F1 (e.g. a squeezing or pinching action).

In relation to such retracted positioning of the grip member 30 relative to the shaft member 20, the pawls 55a and 55b of the second member 50 of the grip member 30 may interface with the rack 24 of the shaft member 20 in a rachet-like manner, wherein the grip member 30 may be retained in the second position shown in FIG. 4B. In this regard, the retracted positioning of grip member 30 is illustrated in FIG. 3B. As shown, the pawls 55a and 55b are disposed in opposing engaged relation with the teeth 22 of the rack 24. By virtue of the retracted positioning of the grip member 30, biasing member 32 may be compressed proximal to pin 36, wherein a distally-directed biasing force may applied by the biasing member 32 to the grip member 30.

To release the grip member 30 from the retracted position, the flange portion 52 of the second member 50 may be distally advanced relative to the flange portion 42 of the first member 40. In turn, upon such advancement a tapered leading edge surface at the distal end of each of the leg members 54a and 54b may interface with opposingly-tapered, leading edge surfaces provided about an inward ledge 48 within the aperture 42 of the first member 40. Upon advancement of the flange portion 52 of the second member 50 relative to the flange portion 42 of the first member 40, the tapered leading edge surface at the distal end of each of the leg member 54a and 54b may advance distally with respect to the inward ledge having opposing-tapered surfaces. Accordingly, the relative distal movement of the leading edge surfaces of the leg members 54a and 54b may result in a lateral movement of the leg members 54a and 54b with respect to the shaft member. That is, the interface of the noted tapered surfaces forces each of the leg members 54a and 54b to elastically deflect outward to a sufficient degree to permit pawls 55a and 55b to disengage from the teeth 22 of the rack 24, wherein the biasing force of biasing member 32 automatically returns the grip member 30 from the retracted position shown in FIG. 4B back to the initial first position shown on FIG. 4C.

To realize tissue release by first and second jaw members 52a and 52b, fingers F1, F2 and F3 of a user may be positioned as shown in FIG. 4C. Then, finger F1 may be advanced relative to fingers F2 and F3 and/or fingers F2 and F3 may be retracted relative to finger F1 so as to release the rachet interface (e.g. a squeezing or pinching action), wherein the biasing member 32 functions to automatically advance the grip member 30 so as to open the first and second jaw members 82 and 84.

In relation to the described endoscopic tool embodiment 1, the tissue grasper 80 may comprise known arrangements in which opposing jaw members pivot between open/closed positions in response to the advancement/retraction or retraction/advancement of an interconnection member that extends to a handle operable by a user. Optionally, a novel tissue grasper may be employed.

For instance, in conjunction with the endoscopic tool described in FIGS. 1-5B, a novel tissue grasper 80 disposed at a distal end of the elongate member 70 may also be provided as shown in FIG. 1. Manipulation of the tissue grasper 80 at the distal end of the elongate member 70 may be realized through relative movement of the inner member 72 and outer member 74.

For instance, the inner member 72 and the outer member 74 may represent a first elongate member and a second elongate member, respectively that are moveable with respect to one another. For purposes of illustration, continued reference will be made to a first elongate member and a second elongate member. However, in various embodiments, the inner member 72 may be the first elongate member or second elongate member and the outer member 74 may be the other of the first elongate member or second elongate member. Therefore, reference to a first or second elongate member is not intended to be limited to either the inner member 72 or outer member 74, unless otherwise specified.

In this regard, the first and second elongate member may be moved between a first and second relative position (e.g., by manipulating the handle 10). In addition, the first jaw member 82 and the second jaw member 84 may move between an open and a closed position. When in the closed position, tissue may be retained and grasped between the first jaw member 82 and the second jaw member 84, and when in the open position the first jaw member 82 and the second jaw member 84 may be generally capable of grasping tissue lying within, intersecting, or adjacent to a grasping plane as will be described further below. In different implementations, the open position and the closed position of the first jaw 82 and the second jaw 84 may correspond with a first relative position of the first and the second elongate members or a second relative position of the first and the second elongate members.

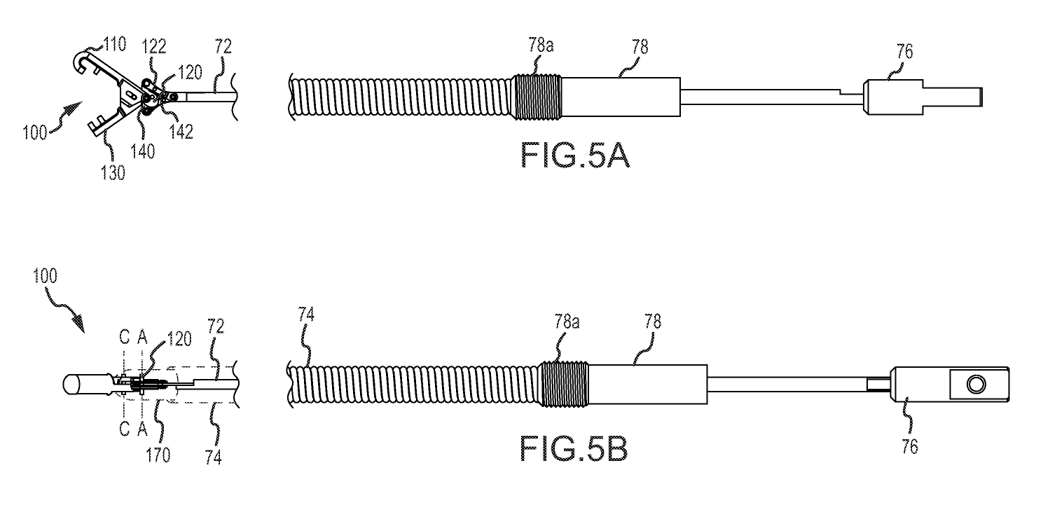

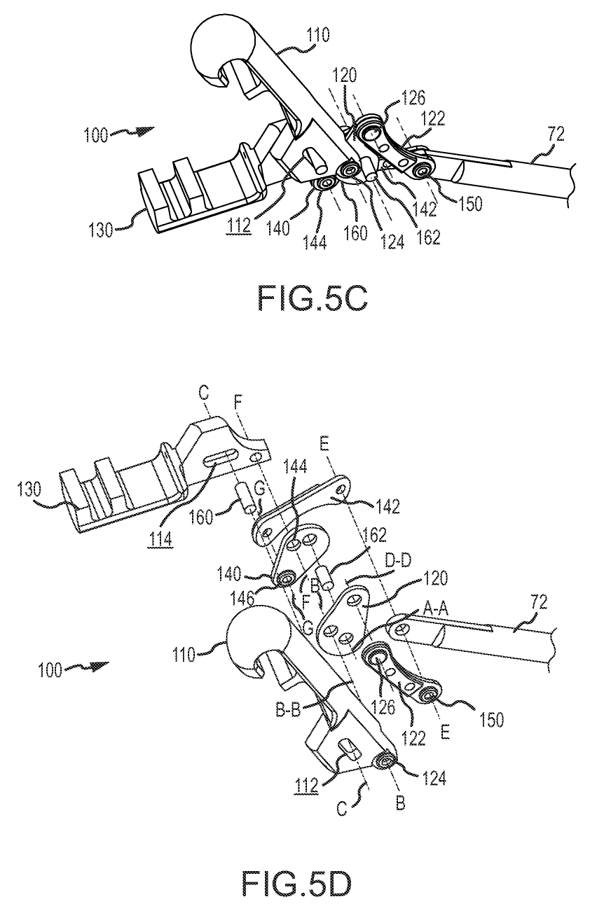

Another embodiment of such a tissue grasper 100 is shown in FIGS. 5A-5D. The tissue grasper 100 may also be employed in various arrangements other than in combination with the embodiment of endoscopic tool 1 described hereinabove.

For purposes of illustration, however, FIGS. 5A-D show the tissue grasper 100 interconnected to the distal end of the elongate member 70 of the endoscopic tool 1 described hereinabove. To facilitate illustration of the tissue grasper 100, a yolk member 170 thereof is not shown in FIGS. 5A, 5C and 5D, nor is the outer member 74 of the elongate member 70 to which the yolk member may be fixedly interconnected. The yolk member 170 is shown in broken lines in FIG. 5B, along with a portion of the outer member 74. Such components and their interconnection will be addressed hereinbelow.

Tissue grasper 100 may generally be moved between an open position and a closed position. The open position and the closed position of tissue grasper 100 may correspond to the first relative position and the second relative position of the first and the second elongate members (e.g., inner member 72 and outer member 74). Furthermore, as shown in FIG. 5B, a yoke 170 may engage portions of the tissue grasper 100. In this regard, the tissue grasper 100 may be provided in a fixed spatial relation relative to the distal end of one of the elongate members (e.g., the first or the second elongate member). For instance, the yoke 170 may act to maintain portions of the tissue grasper 100 in a fixed spatial relation relative to the distal portion of either the first or the second elongate members.

A first jaw member 110 and an opposing second jaw member 130 having opposing shaped surfaces may be provided. The first jaw member 110 may be interconnected to the distal end of either the first or the second elongate member via a first articulating member 120 and a second articulating or linkage member 122. Similarly, a second jaw member 130 may be interconnected to the distal end of inner member 72 via a first or second articulating member 140 and a second articulating or second linkage member 142. As will be described, the first and second linkage members 122, 142 translate or communicate, movement of a distal end of at least one of the first and second elongate members to the first and second articulating members 120, 140, respectively.

The first articulating member 120 may be moveable with respect to an axis A-A. In this regard, axis A-A may be maintained in a fixed spatial relation relative to one of the ends of the first or second elongate members. For instance, the axis A-A may coincide with a shaft 162 which is engaged by the yoke 170 and thereby maintained in a fixed spatial relation relative to the distal end of one of the elongate members. That is, the yoke 170 may be interconnected to either the first or second elongate member, the axis A-A may be maintained in a fixed spatial relation relative to the interconnected first or the second elongate member. As may be appreciated, since axis A-A may be positioned in fixed spatial relation relative to one of the first or the second elongate members, the other of the first and second elongate members (e.g., to which axis A-A is not in a fixed spatial relation) may engage the articulating member 120 to produce movement thereof relative to axis A-A.

In this regard, the movement of the articulating member 120 may define a first path between a first articulated position and a second articulated position that may correspond to movement of the first and the second elongate members between the first relative position and the second relative position. In one embodiment, the first articulating member 120 may be rotatable with respect to axis A-A. As such, at least a portion of the first path may be arcuate between the first and the second articulated positions. That is, any one fixed point on the articulating member 120 may move on an arc between the first and the second articulated positions. Thus, movement of the first articulated member beyond the first or the second articulated positions (that is, outside the range of motion between the first and second articulated position) may not be movement of the first articulated member along the first path. Stated differently, the first path may not extend beyond a range between the first articulated position and the second articulated position. In addition, the first articulated position and the second articulated position may further correspond to the open position and the closed position of the first jaw member 110.

The first jaw member 110 may be moveable with respect to the first articulating member 120 relative to an axis B-B that intersects both the first jaw member and the first articulating member 120. In one embodiment, rotation of the first articulating member 120 about axis A-A may also result in movement of the first jaw member 110 with respect to the first articulating member 120 at axis B-B.

The first articulating member 120 may rotate in a first direction when moving from the first articulated position to the second articulated position and the second direction when moving from the second articulated position to the first articulated position. As the first or the second articulated position may be associated with the open position and the other of the first or the second articulated position may correspond to the closed position of the first jaw member 110, rotation of the first articulating member 120 along the first path between the first articulated position and the second articulated position may result in the first jaw member 110 moving between the open position and the closed position.

Additionally, the first jaw member 110 may also move with respect to an axis C-C. Axis C-C may also be located in a fixed spatial relation relative to the distal end of one of the first or the second elongate members. In this regard, axis C-C may coincide with a shaft 160 which is engaged by the yoke 170. As the yoke 170 is disposed at the distal end of one of the first or the second elongate members, axis C-C may also be at a fixed spatial relation relative to the distal end of one of the elongate members. In this regard, axis A-A and axis C-C may be arranged in a fixed spatial relation relative to one another. That is, axis A-A and axis C-C may be spaced apart or offset.

Movement of the first articulating member 120 between the first articulated position and the second articulated position and the corresponding movement of the first jaw member 110 between the open position and the closed position may result in the first jaw member 110 moving with respect to axis C-C. For instance, jaw member 110 may pivot with respect to axis C-C in order to move from the open position to the closed position.

The first articulating member 120 may also be moveable with respect to a first linkage member 122. The first linkage member 122 may be moveable with respect to an axis D-D that also intersects the first articulating member 120 such that the first articulating member may move with respect to axis D-D. The first linkage member 122 may also move with respect to an axis E-E that intersects the distal end of one of the first or the second elongate members. For illustrative purposes, the first linkage member 122 is shown as being moveable with respect to the inner member 72 at axis E-E, yet as discussed above the first linkage member 122 could be moveable at axis E-E with respect to the outer member 74 (e.g., in a case where the yoke 170 was attached to a distal end of the inner member). In this regard, the first linkage member 122 may move when the first and the second elongate members are moved between their first relative position and the second relative position. That is, axis E-E may move with respect to axis A-A and axis C-C upon movement of the first and the second elongate members between the first relative position and the second relative position.

In one embodiment, the first linkage member 122 is curved along a length between the axis E-E and the axis D-D. For instance, the first linkage member 122 may generally curve away from a longitudinal axis of the distal end of the first or second elongate members. In this regard, axis D-D may be offset from axis E-E such that axis D-D is offset from the longitudinal axis of the distal end of the first or second elongate members. In this regard, the first linkage member 122 may include a portion at an end of the first linkage member 122 that is movably connected to the first articulating member 120 at axis D-D such that the end portion of the first linkage member 122 may be substantially perpendicular with respect to axis A-A and axis D-D at some point during the movement of the tissue grasper 100 between the closed and open positions.

In one embodiment, the first and the second elongate member may be disposed for axial movement with respect to one another. In this regard, the first and the second relative positions may correspond to a first relative axial position and a second relative axial position between the distal ends of the first and the second elongate members. The axial position associated with the first relative position may be offset from the axial position associated with the second relative position. In other words, axis E-E may move with respect to axis A-A such that axis E-E and axis A-A are disposed at a first axial relative position when the first and the second elongate members are in the first relative position and axis A-A and axis E-E may be disposed at a second axial relative position when the first and the second members are disposed in the second relative position. In this regard, axis A-A and axis E-E may move axially with respect to one another in response to axial movement of the first and the second elongate members between the first and the second relative position.

In any case, the first linkage member 122 may be moveable with respect to axis E-E and axis D-D. The first linkage member 122 may rotate with respect to axis E-E and may in turn cause the first articulating member 120 to rotate between the first articulated position and the second articulated position along the first path. In this regard, the first linkage member 122 may rotate with respect to the first articulating member 120 and axis D-D. As the first articulating member 120 rotates between the first articulated position and the second articulated position, the movement of the first articulating member 120 may also result in movement of the first jaw member 110 with respect to axis B-B. In fact, axis B-B may move from a first offset position to a second offset position. That is, when the first articulating member 120 is at the first articulated position, the axis B-B may be in the first offset position. As the first articulating member 120 moves between the first articulated position and the second articulated position, axis B-B may move therewith. Thus, when the first articulating member 120 is in the second articulated position, axis B-B may be in the second offset position. The first and the second offset positions may be offset.

Additionally, the embodiment depicted in FIG. 5C may include a second jaw member 130 moveable between an open and a closed position. Furthermore, a second articulating member 140 may be provided that is also moveable with respect to axis A-A. The second articulating member 140 may be moveable between a third articulated position and a fourth articulated positions that corresponds with the movement of the first and the second elongate members between the first and the second relative position. For instance, the second articulating member 140 may move between the third and the fourth articulated positions with respect to axis A-A. Accordingly, the second articulating member 140 may move along a second path between the third and the fourth articulated positions. Thus, movement of the second articulated member beyond the third or the fourth articulated positions (that is, outside the range of motion between the third and fourth articulated position) may not be movement of the second articulated member along the second path. Stated differently, the second path may not extend beyond a range between the third articulated position and the fourth articulated position. In one embodiment, the second articulated member 140 may pivot with respect to axis A-A. As such, the second path may be at least partially arcuate.

The second jaw member 130 may move with respect to an axis F-F that also intersects the second articulating member 140. In one embodiment, the second jaw member 130 may pivot with respect to axis F-F. Axis F-F may move commensurately with the second articulating member 140 as the second articulating member 140 moves between the third and the fourth articulated position. In this regard, axis F-F may be at the third offset position when the second articulating member 140 is in a third articulated position and axis F-F may be in a fourth offset position when the second articulated member 140 is in the fourth articulated position.

The second articulating member 140 may also be moveable with respect to a second linkage member 142. The second linkage member 142 may also move with respect to an axis G-G that also intersects the second articulating member 140. In one embodiment, the second articulating member 140 may pivot with respect to the axis G-G. The second linkage member 142 may move with respect to axis E-E.

In one embodiment, the second linkage member 142 is curved along a length between the axis E-E and the axis F-F. For instance, the second linkage member 142 may generally curve away from a longitudinal axis of the distal end of the first or second elongate members. In this regard, axis F-F may be offset from axis E-E such that axis F-F is offset from the longitudinal axis of the distal end of the first or second elongate members. In this regard, the second linkage member 142 may include a portion at an end of the second linkage member 142 that is movably connected to the second articulating member 140 at axis F-F such that the end portion of the second linkage member 142 may be substantially perpendicular with respect to axis A-A and axis F-F at some point during the movement of the tissue grasper 100 between the closed and open positions.

In this regard, as axis E-E is moved with respect to axis A-A, the second linkage member 142 may move with respect to axis E-E as well as axis G-G. The movement of axis G-G may also result in movement of the second articulating member 140 between the third and the fourth articulated position. As the second articulating member 140 moves between the third and the fourth articulated position, movement of the second articulating member 140 about axis F-F may also impose movement of the second jaw member 130 about axis F-F. In this regard, as articulating member 140 moves between the third and the fourth position the second jaw member 130 may move between the open and the closed position. As such, similar to the movement of the components discussed with respect to the first jaw member 110, the second jaw member 130 may be moved between the open and the closed position corresponding to movement of the second articulating member 140 between the third and the fourth articulated position and also corresponding to the movement of the first and the second elongate member between the first and the second relative position.

The second jaw member 130 may also move with respect to axis C-C when moving between the open and the closed position. Like the first jaw member 110, the second jaw member 130 may pivot with respect to axis C-C when moving from the open to the closed position.

With continued reference to FIGS. 5A-5D, but stated differently, the first articulating member 120 may be pivotably interconnected to the first jaw member 110 via pivot member 124 (located on axis B-B), wherein the first jaw member 110 and the first articulating member 120 may each pivot relative to the pivot member 124. The pivot member 124 may be disposed on the first articulating member 120 such that the pivot member 124 (and axis B-B) are at a first angular position with respect to axis A-A when the first articulating member 120 is in the first articulated position and at a second angular position with respect to axis A-A when the first articulating member is in the second articulated position. The first articulating member 120 may be pivotably interconnected to the first linkage member 122 via pivot member 126 (located on axis D-D), wherein the first articulating member 120 and the first linking member 122 may each pivot relative to the pivot member 126.

Further, the first linkage member 122 may be pivotably interconnected to the distal end of the first or the second elongate member. In one embodiment, the first linkage member 122 may be pivotably interconnected to the inner member 72 via a pivot member 150 (located on axis E-E), wherein the first linkage member 122 may pivot relative to the pivot member 150 (located on axis E-E).

In like fashion, the second jaw member 130 may be pivotably interconnected to the second articulating member 140 via pivot member 144 (located on axis F-F), wherein the second jaw member 130 and second articulating member 140 may each pivot relative to the pivot member 144.

The second articulating member 140 may be pivotably interconnected to the second linkage member 142 via pivot member 146 (located on axis (G-G). In one embodiment, the second articulating member 140 and the second linkage member 142 may pivot with respect to one another at an axis G-G. The first articulating member 140 and the second linkage member 142 may each pivot relative to the pivot member 146. Further, the second linkage member 142 may be pivotably interconnected to the distal end of one of the first or the second elongate members. In one embodiment, the second linkage member 142 is connected to the inner member 72 via a pivot member 150, wherein the second linkage member 142 may pivot relative to the pivot member 150.

As shown in FIG. 5C the first jaw member 110 may include a slot 112 passing therethrough. Similarly, as shown in FIG. 5D the second jaw member 130 may be provided with a slot 114 passing therethrough. In turn, the tissue grasper 100 may further include a first shaft 160 (coinciding with axis C-C) fixedly interconnected to a yolk member (not shown in FIGS. 5A, 5C, and 5D). The yolk member 170 may be fixedly interconnected to the distal end of the first or the second elongate member. For instance, as shown, the yolk member 170 may be interconnected to the distal end of the external member 74 of the endoscopic tool 1. The first shaft 160 may be disposed through the slot 112 of the first jaw member 110 and the slot 114 of the second jaw member 130. As will become apparent herein below, first jaw member 110 and the second jaw member 130 may move relative to the first shaft member 160 in response to movement of the first elongate member with respect to the second elongate member and of the rotation of the first articulating member 120 and the second articulating member 140, respectively. In the illustrated approach, slot 112 of the first jaw member 110 and slot 114 of the second jaw member 130 may slide relative to the first shaft 160. In addition, the first jaw member 110 and second jaw member 130 may also pivot with respect to the first shaft 160. Accordingly, the first and second jaw members 110 and 130 may pivotally and slidingly engage the first shaft 160. In turn, and as will be further described, the distance between the first shaft 160 and distal ends of first and second jaw members 110, 130 is less in the closed jaw position than in the open jaw position. Conversely, a distance between the first shaft 160 and the pivot member 124 is greater in the closed jaw position than the open jaw position. As may be appreciated, such arrangement facilitates the realization of increased grasping forces in response to a user's manipulation of handle 10 to effect relative movement of the distal ends of the first elongate member and second elongate member.

As further illustrated by FIGS. 5C, the tissue grasper 100 may further include a second shaft 162 coinciding with axis A-A extending through each of the first and second articulating members 120 and 140. In turn, the second shaft 162 may be interconnected to the referenced yolk member of the tissue grasper 100 (not shown in FIG. 5C), wherein such yolk member may be fixedly interconnected to the distal end of the external member 74 of the endoscopic tool 1. Accordingly, the first shaft 160 and the second shaft 162 may be arranged in a fixed apart fashion and held thusly by the yolk member (not shown). As it will become apparent hereinbelow, the first articulating member 120 and the second articulating members 140 may each pivot about the second shaft 162 in response to advancement/retraction of the inner member 72.

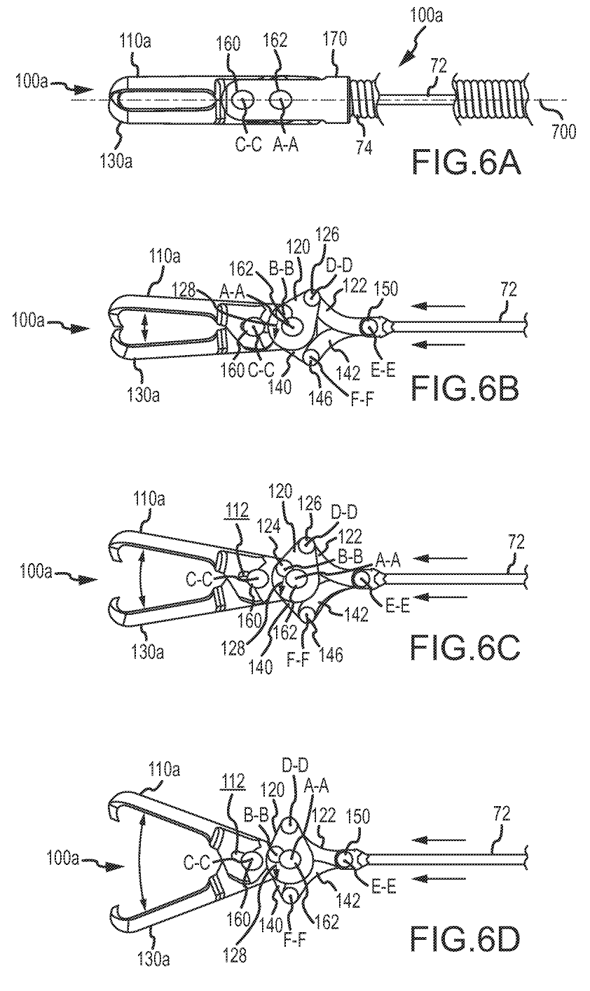

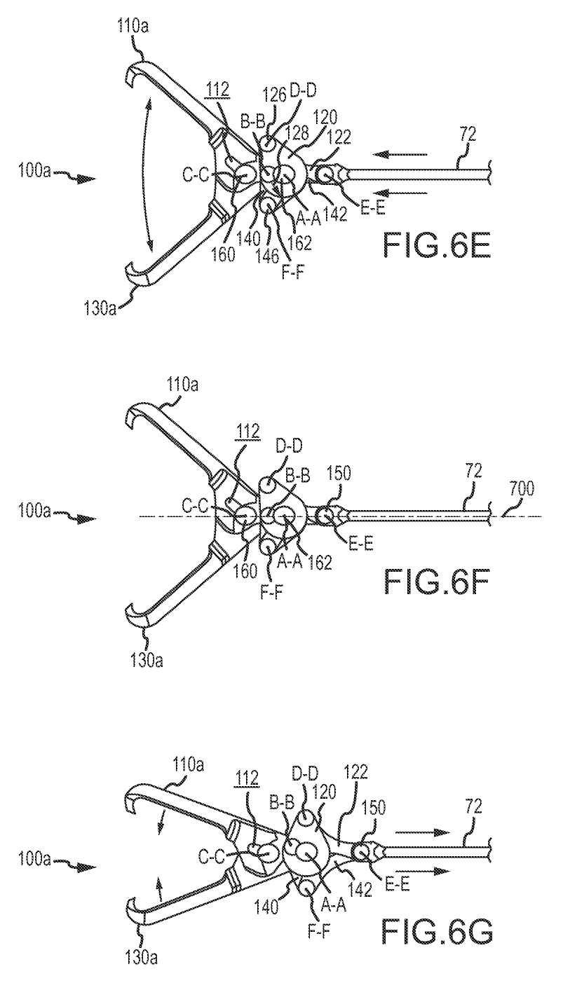

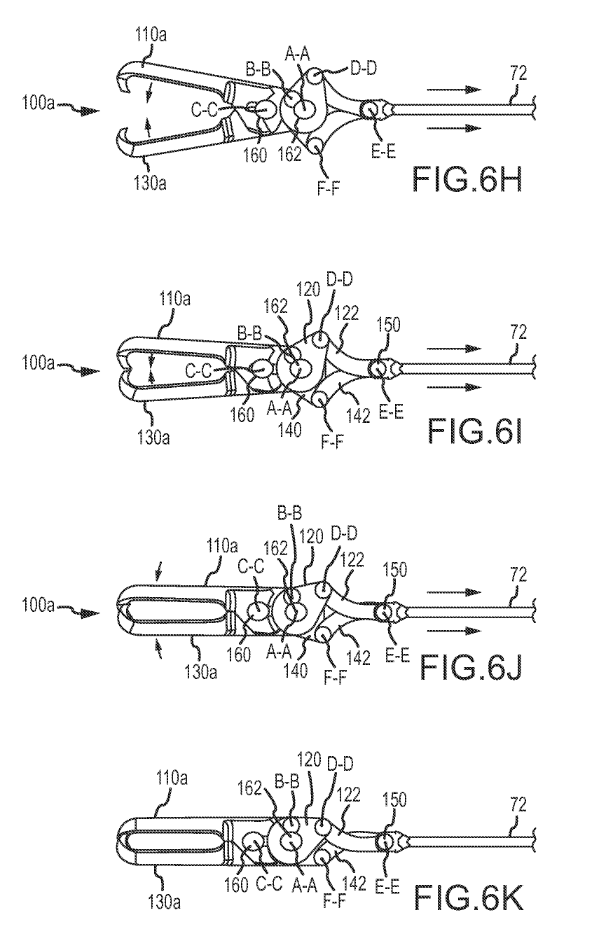

Reference is now made to FIGS. 6A-6K, which illustrate the opening and the closing of a modified tissue grasper 100a. In this regard, the modified tissue grasper 100a may be of the same construction as tissue grasper 100 described hereinabove, with the exception that the opposing face portions of the first jaw member 110 and second jaw member 130 have been modified as illustrated. In turn, the reference numerals utilized in relation to the description associated with FIGS. 5A-5C are also utilized in relation to FIGS. 6A-6K.

As shown in FIG. 6A, the tissue grasper 100a may include a yoke member 170 affixed to an outer member 74. For purposes of illustration, the yoke 170 may be affixed to the outer member 74 such that axis A-A and axis C-C are in a fixed spatial relation relative to the outer member 74. In conjunction, the inner member 72 may be in a fixed spatial relation relative to axis E-E. That is, yoke 170 may be provided in a fixed spatial relation between the distal end of the outer member 74 and axis A-A and axis C-C while inner member 72 may be provided in a fixed spatial relation with axis E-E. Thus, movement of axis E-E with respect to axis A-A and axis C-C may be facilitated.

As shown in FIG. 6B, the first elongate member and second elongate member may have begun to move between the first relative positions to the second relative position. This may result in the first linkage member 122 moving with respect to axis E-E and axis D-D. Additionally, axis E-E may move relative to axis A-A. In FIG. 6B, the relative movement of axis E-E to axis A-A may cause the first linkage member 122 to initiate rotation of the first articulating member 120 from the first articulated position towards the second articulated position along the first path. The first articulating member 120 may rotate along the first path between the first articulated position and the second articulated position with respect to axis A-A and in turn the first jaw member 110a may begin to move from the closed position to the open position by moving with respect to axis B-B as the first articulating member also moves with respect to axis B-B.

FIG. 6C depicts the tissue grasper 100a as the first and the second elongate members have progressed further between the first relative position and the second relative position as was shown in FIG. 6B. Axis E-E may continue to move with respect to axis A-A such that the first articulating member 120 continues to rotate about axis A-A along the first path in response to movement of the first and the second elongate members from the first relative position to the second relative position. Additionally, the first articulating member 120 may continue to move such that the position of axis B-B moves toward the second offset position which, in turn, may result in continued movement of the first jaw member 100a from the closed toward the open position. As the first and the second elongate members continue to move from the first relative position to the second relative position, the movements described above may continue as shown in FIGS. 6D and 6E until at FIG. 6F the first and the second elongate members have moved completely from the first relative position to the second relative position such that the first articulating member 120 has moved from the first articulated position to the second articulated position and the first jaw member 110a has moved from the closed position to a the open position.

In other words, the first and the second linkage members 122 and 142 may be at least partially laterally advanced with respect to the second shaft 162. Accordingly, a tangential force component 128 may be imparted on the first and the second articulating members 120 and 140 along the first and the second paths, such that the first and the second articulating members 120 and 140 may be rotated about the second shaft 162 along the first and the second paths, respectively. The pivot members 124 and 144 may be rotated along with the first articulating member 120 and the second articulating member 140, respectively. This may cause the jaw members 110a and 130a to move with respect to the first shaft 160 (e.g., pivot about the first shaft 160). Accordingly, the jaw members 110a and 130a move relative to the first shaft 160 such that the first shaft 160 moves laterally along the slots 112 and 114 while the jaw member 110a and 130a also pivot about the first shaft 160. Accordingly, the jaw members 110a and 130a are progressively moved to the open position.

Reference is now made to FIG. 6F which shows the tissue grasper 100a in the open position. In such position, the slot 112 of the first jaw member 110a and slot of the second jaw member 130a have advanced relative to first shaft member 160. In such position, the tissue grasper 100a may be located for grasping a tissue region of interest.

The embodiment depicted in FIGS. 6A and 6F may have a grasping plane 700. The grasping plane 700 generally may be arranged such that axis A-A, C-C, and E-E are all disposed within in the grasping plane 700. As can be seen in FIG. 6F, when in the open position the jaw members 110a, 130a may be placed adjacent to tissue that lies on, adjacent to, or intersects the grasping plane 700 such that when the jaw members are in the closed positioned as shown in FIG. 6A the tissue may be retained between the jaw members 110a, 130a. It should be noted that the axis A-A, C-C, and E-E may be lie within the grasping plane 700 in both the open and the closed position.

Moreover, the axis A-A, C-C, and E-E may also be orthogonal relative to and intersecting a longitudinal axis of a distal end of one of the first or second elongate members. For instance, the axis A-A, C-C and E-E may be orthogonal relative to and intersecting a longitudinal axis of the internal member 72.

Reference is now made to FIGS. 6F-6K which progressively illustrate closure of the first and the second jaw members 110a and 130a in response to relative movement of first and the second elongate members. The yolk member 170 is not shown for purposes of clarity. FIG. 6K illustrates the tissue grasper 100a in the closed position, wherein slot 112 of first jaw member 110a and the slot of the second jaw member 130a are retracted relative to first shaft 160.

FIGS. 6G, 6H, 6I and 6J show a progression as the first and the second elongate members move from the second position toward the first position such that the first articulating member 120 moves from the second articulated position to the first articulated position along the first path and the first jaw member 110a moves from the open position to the closed position along the second path. During this progression, axis E-E may move with respect to axis A-A in a direction opposite of the direction in which axis E-E moved during the opening shown in FIGS. 6A-6E. Additionally, the articulating member 120 may move in a direction along the first path between the first articulating member and the second articulated position in the direction opposite that which it traveled when moving from the first articulated position to the second articulated position. Similarly, the first jaw member 110a may move in the opposite direction from the open position to the closed position as it did from the closed position to the open position.

In this regard, movement of the first and the second elongate members from the first relative position to the second relative position may result in the first articulating member 120 moving from the first articulated position to the second articulated position, the second articulating member 140 moving from the third articulated position to the fourth articulated position, the second jaw member 130a moving from the closed position to the open position, and the first jaw member 110a moving from the closed position to the open position. Conversely, movement of the first and the second elongate members from the second relative position to the first relative position may result in movement of the first articulating member 120 from the second articulated position to the first articulated position, the second articulating member 140 moving from the fourth articulated position to the third articulated position, the second jaw member 130a moving from the open position to the closed position, and the first jaw member 110a moving from the open position to the closed position.

The closed positioning of the tissue grasper 100a may typically be employed during positioning of the tissue grasper 100a relative to a tissue region of interest. As shown in FIG. 6A, tissue grasper 100a may include a yolk member 170, as a referenced above. The yolk member 170 may include a proximal end portion 172 for fixed interconnection to one of the first or the second elongate members (e.g., the external member 74 (not shown) of the endoscopic tool 1). As shown in FIG. 6A, first shaft 160 and the second shaft 162 may be interconnected to the yolk member 170.

As may be appreciated, the first and second jaw members 110a and 130a are effectively locked in the closed position shown in FIG. 6K by virtue of the illustrated and described arrangement.

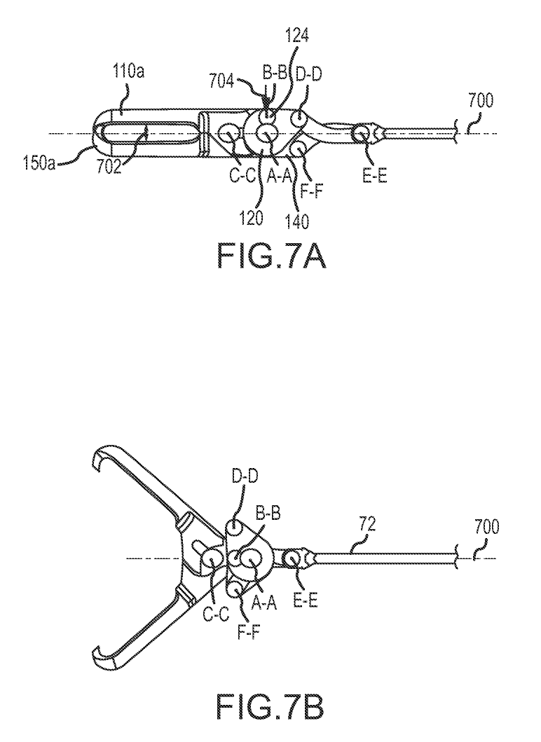

FIGS. 7A and 7B show the tissue grasper 100a in closed position and a fully opened position, respectively. In FIG. 7A, the tissue grasper 100a may be effectively locked such that a separating force 702 acting one the first jaw member 110a or second jaw member 130a may be resisted. The separating force 702 may include a force acting normally to the opposing face portions of the jaw member 110a and 130a. Thus, the separating force 702 may at least be partially directed in a manner with respect to the first jaw member 110a or second jaw member 130a such that the jaw member are urged toward the open position. As described above, movement of the first jaw member 110a from the closed position to the open position results in a corresponding movement of the first articulating member between the first and the second articulated positions. As depicted in FIG. 7A, the separating force 702 may act on the first jaw member 110a yet no force may be affected to the first articulating member 120 that will result in movement of the first articulating member along the first path (i.e., between the first articulated position shown in FIG. 7A and the second articulated position shown in FIG. 7B).

As such, the first jaw member 110a will not move from the closed position to the open position upon application of the separating force 702 to the jaw member 110a when the tissue grasper 100a is in the closed position as shown in FIG. 7A. This may be due in part to lack of movement of the first articulating member 120 as no force is not acting on the first articulating member 120 to move the first articulating member 120 along the first path between the first and the second articulated position. For example, in FIG. 7A, a separating force 702 acting on the first jaw member 110a may tend to cause the first jaw member 110a to attempt to pivot with respect to axis C-C. A rotation about axis C-C may also correspond in force acting at axis B-B with respect to axis A-A.

However, in FIG. 7A, all force transferred from the separating force 702 acting on the first jaw member 110a may result in axis B-B simply being urged towards axis A-A as shown by the resulting force 704. That is, no resulting tangential force (e.g., force 128 as shown in FIGS. 6B-6E) may be affected to the first articulating member 120. Thus, the articulating member 120 may not be rotated between the first and the second articulated position. That is, based on the arrangement of the first articulating member 120 with respect to the first jaw member 110a, any resulting force 704 acting between axis B-B and axis A-A may be directed along the direction between axis A-A and B-B as shown in FIG. 7A.

As stated above, movement of the first jaw member 110a includes a corresponding movement of the first articulating member 120. In FIG. 7A, the separating force 702 acting on jaw member 110a does not result in force acting on the first articulating member 120 at axis B-B along the first path. That is, the spatial arrangement of the first articulating member 120 with respect to the first jaw member 110a does not result in rotation upon receiving a separating force 702 on the first jaw member 110a because no force acts on the first articulating member 120 along the first path (e.g., tangentially to the first articulating member 120 with respect to axis A-A).

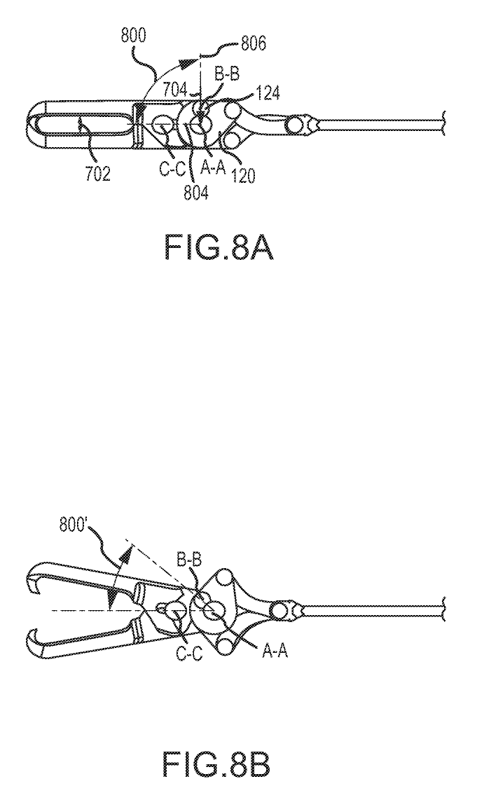

As shown in FIG. 8A, the arrangement of axis C-C, axis A-A and axis B-B may include an angle 800 therebetween that is substantially a 90° angle when the tissue grasper 100a is in the closed position. In so much as the angle 800 formed by axis C-C, axis A-A, and axis B-B forms at least a 90 degree angle, the any resulting force vector 704 acting on the first articulating member 120 at axis B-B may be directed toward axis A-A such that no component of the vector extends in a tangential direction with respect to the first articulating member 120 and axis A-A. As no tangential force acts on the articulating member 120, as is required to move the articulating member 120 along the first path between the first articulated member and the second articulated member, the tissue grasper assembly 100a in turn may not move between the closed and the open position. That is, movement between the closed and the open position of the jaw member 110a is effectively resisted because no force results from an application of the separation force on 110a onto the first articulating member 120 tending to move the articulating member 120 along the first path between the first and the second articulated position.

Stated differently, the opening force 702 may be transferred to pivot member 124. The result may be a resultant force 704 acting on the first articulating member 120 at the pivot member 124. Because when in closed position as shown in FIG. 8A, the first angular position (represented by the axis 806) of the pivot member 124 may be substantially perpendicular to an axis segment 804 defined between the centerlines of the first shaft 160 and the second shaft 162, any resulting force 704 acting on the pivot member 124 may be directed perpendicularly to the axis segment 804 and in a radial direction with respect to the first articulating members 120. That is, the resulting force 704 acts substantially radially on the first articulating member 120. As no tangential force component acts on the first articulating member 120 as a result of the arrangement of the components, the first articulating member 120 may not rotate in response to the application of the opening forces 702 to the first jaw member 110a.

Alternatively, the angle 800 may be greater than 90 degrees As such, a resultant force 704 acting on the first or the second articulating members 120 or 140 may cause the first articulating member 120 to receive a tangential force in a direction not along the first path. However, any further rotation of the first articulating member 120 beyond the first path may be impeded or prevented by interference between the first jaw member 110a and the second shaft 162. Accordingly, any resultant tangential force acting with respect to the first articulating member 120 may be resisted as the first jaw member 110a interferes with the second shaft 162 such that any rotation of the first articulating member 162 is resisted. Furthermore, as the resulting tangential force acting on the first articulating member 120 when the angle 800 is greater than 90 degrees urges the first articulating member 120 away from the second articulated position, the resultant force does not act along the first path. As stated above, the first path may run from the first articulated position to the second articulated position. As the first articulating member 120 may be in the first articulated position as shown in FIG. 8A, any resultant tangential force when the angle 800 is greater than 90 degrees will urge the first articulating member 120 away from the second articulated position. Thus, the resultant tangential force will not act along the first path spanning between the first articulated position and the second articulated position.

However, when the tissue grasper 100a is in the open position or is between the open position and the closed position, axis B-B, axis A-A, and axis C-C may form an included angle 800′ of less than 90 degrees. Thus, the tissue grasper 100a may not be locked as a resulting force may include a force vector urging the first articulating member 120 along the first path. Thus, when the included angle 800′ between axis B-B, axis A-A, and axis C-C is less than 90 degrees, the tissue grasper may not be in a locked position.

While the above locking of the tissue grasper 100a has been primarily discussed with regard to the first articulating member 120 resisting an opening force, it is to be understood that the same principles apply with respect to the second articulating member 140. That is, axis A-A, axis C-C, and axis F-F may form at least a 90 degree angle when in the closed position such that no tangential force results from a separating force 702 on the second articulating member to cause it to move in the second path. Thus, the second jaw member 130a may also resist movement to the open position when in the closed position.

As noted above, it will be appreciated that when in the closed arrangement, the transfer of forces resulting from the relative movement of the first and second elongate member may be greater when the tissue grasper 100a is in the closed jaw position than when in the open jaw position. This may result from changing dimensions of lever arms. For instance, a first lever arm may be defined between axis B-B and axis C-C. Movement of this lever arm may effect and thereby correspond to movement of the first jaw member 110a including a distal end thereof. Accordingly a second lever arm may be defined from the distal end of the first jaw member 110a to axis C-C.

When in the closed jaw position, a length of the first lever arm may be greater than when in the open jaw position. Conversely, the length of the second lever arm may be less when in the closed jaw position than when in the open jaw position. Accordingly, the ratio of the first lever arm to the second lever arm may be greater when in the closed jaw position than when in the open jaw position. As such, the mechanical advantage realized by the lever arms may be greater when in the closed jaw position than in the open jaw position.

Similarly, the second jaw member 130a may have similar characteristics. That is, a third lever arm may extend from axis F-F to axis C-C. The length of this lever arm may be greater when in the closed jaw position than when in the open jaw position. A corresponding fourth lever arm having a distance from a distal end of the second jaw member 130a to axis C-C may be shorter when in the closed jaw position than when in the open jaw position. Accordingly, a ratio between the third and forth lever arms may be greater when in the closed jaw position such that the mechanical advantage realized may be greater when the second jaw member 130a is in the closed jaw position than when in the open jaw position.

The embodiment descriptions provided hereinabove are strictly for purposes of illustration and are not intended to limit the scope of the present invention. Modifications, additions and adaptations will be apparent to those skilled in the art and are intended to be within the scope of the present invention.

1. An endoscopic tool for selective tissue grasping and retention, comprising:
a flexible elongate member including a tubular outer member and an inner member extending through said outer member, wherein said internal member and said outer members are disposed for relative movement therebetween;
a tissue grasper located at a distal end of said elongate member and including a first jaw member and a second jaw member, wherein opposing portions of said first and second jaw members are disposed for relative movement between an open position and a closed position, wherein tissue is graspable between said first and second jaw members in said closed position;
a handle located at a proximal end of said elongate member and including a shaft member and a grip member extending about at least a portion of said shaft member, said shaft member and said grip member being disposed for selective relative movement by user along a handle axis, wherein one of said shaft member and said grip member includes a pawl and the other of said shaft member and said grip member includes a rack comprising a plurality of teeth spaced along said handle axis to define a rachet interface therebetween; and,
wherein first relative movement between said grip member and said shaft member by a user affects first relative movement between said inner member and said outer member of said elongate member and first relative movement between said opposing portions of said first and second jaw members of the tissue grasper from said open position to said closed position, and wherein said rachet interface retains said opposing portions of said first and second jaw members in said closed position.

2. An endoscopic tool as recited in claim 1, wherein said handle axis is linear.

3. An endoscopic tool as recited in claim 1, wherein one of said pawl and said rack is selectively moveable laterally away from said other of said pawl and said rack by a user to permit second relative movement between said grip member and said shaft member to affect second relative movement between said inner member and said outer member of the elongate member, and second relative movement between first and second jaw members of the tissue grasper from said closed position to said open position.

4. An endoscopic tool as recited in claim 3,
wherein said selective lateral movement results from a tapered leading edge surface of a first portion of said grip member advancing relative to an opposingly-tapered surface of a second portion of said grip member.

5. An endoscopic tool as recited in claim 4, said handle further comprising:
a biasing member for applying a biasing force to oppose said first relative movement between said grip member and said shaft member, wherein upon said selective lateral movement said biasing force automatically affects said second relative movement between said grip member and said shaft member.

6. An endoscopic tool as recited in claim 5, wherein said first relative movement and said second relative movement between said shaft member and said grip member is completable by a user via one-hand operation of the endoscopic tool.

7. An endoscopic tool as recited in claim 6, said grip member including first and second members interconnectable and disposed for selective relative movement therebetween, wherein first relative movement between said first and second members by a user affects said lateral movement of one of said pawl and said rack away from the other one.

8. An endoscopic tool as recited in claim 7, wherein said first and second members include corresponding first and second lateral flange portions extending away from said handle axis in opposing relation to each other.

9. An endoscopic tool as recited in claim 8, said handle further comprising:
a proximal end member fixedly interconnected to a proximal end of said shaft member, wherein said proximal end member is advanceable by a user relative to said grip member to affect said first relative movement between said shaft member and said grip member.

10. An endoscopic tool as recited in claim 9, wherein said first lateral flange portion is engageable by one or more digits of a user's hand and said proximal end member is engageable by another digit of said user's hand to affect said first relative movement.

11. An endoscopic tool as recited in claim 9, wherein said first lateral flange portion is engageable by one or more digits of a user's hand and said second lateral portion is engageable by another digit of said user's hand to affect said second relative movement.

12-63. (canceled)

64. An endoscopic tool as recited in claim 2, wherein said rachet interface is provided along said linear handle axis.

65. An endoscopic tool as recited in claim 5, wherein said grip member extends at least partially around the shaft member.

66. An endoscopic tool as recited in claim 65, wherein said shaft member extends through an aperture of the grip member.

67. An endoscopic tool as recited in claim 5, wherein said biasing member is positioned within the shaft member.

68. An endoscopic tool as recited in claim 5, wherein said biasing member is compressed upon said first relative movement to apply said biasing force to said grip member.

69. An endoscopic tool as recited in claim 9, wherein one of said inner member and said outer member is interconnected to said shaft member and the other one of said inner member and said outer member is interconnected to said grip member.

70. An endoscopic tool as recited in claim 69, wherein said inner member is interconnected to said grip member for co-movement therewith relative to said shaft member and said proximal end member.

71. An endoscopic tool as recited in claim 70, wherein said grip member extends about said shaft member, and further comprising:
a pin for interconnecting said inner member and said grip member, wherein said pin extends through a slot of said shaft member, and wherein said pin is moveable within and along the slot during relative movement of said shaft member and said grip member.

72. An endoscopic tool as recited in claim 71, wherein said biasing member is positioned within the shaft member.
